(19) United States
(12) Patent Application Publication
(10) Pub. No.: US 2024/0402389 A1
(43) Pub. Date: Dec. 5, 2024

(54) TUNEABLE MXENE-BASED LENS DESIGN FOR SPECIFIC WAVELENGTH FILTRATION TO AID OCULAR DISORDERS AND PROTECT FROM POTENTIAL HARMFUL RADIATION

(71) Applicant: DREXEL UNIVERSITY, Philadelphia, PA (US)

(72) Inventors: Yury GOGOTSI, Warminster, PA (US); Susan Rachel SANDEMAN, Brighton (GB); Emma Jade WARD, Brighton (GB); Maria Natalia NORIEGA PEDRAZA, Brighton (GB)

(21) Appl. No.: 18/693,678

(22) PCT Filed: Sep. 30, 2022
(86) PCT No.: PCT/US2022/077335

§ 371 Date: Mar. 20, 2024

Related U.S. Application Data
(60) Provisional application No. 63/250,361, filed on Sep. 30, 2021.

(51) Int. Cl. G02B 1/04; C08K 3/14; G02C 7/10

(52) U.S. Cl. CPC G02B 1/043; C08K 3/14; G02C 7/104

(57) ABSTRACT

Provided are ophthalmic components (e.g., eyeglass lenses and contact lenses) that include a two dimensional crystalline solid material dispersed in a matrix material. The disclosed components can selectively filter light of selected wavelengths, thereby making the components suitable for use by individuals who may suffer from color vision deficiency conditions. The two dimensional crystalline solid material comprises at least one layer comprising a substantially two-dimensional array of crystal cells; each crystal cell having an empirical formula of M(n+1)X(n), such that each X is positioned within an array of M, wherein M is at least one Group IIIB, IVB, VB, VIB or VIIB metal, and wherein X is C and/or N.

### CROSS-REFERENCE TO RELATED APPLICATIONS

The present application claims priority to and the benefit of U.S. patent application No. 63/250,361, “Tuneable-MXene-Based Lens Design For Specific Wavelength Filtration To Aid Ocular Disorders And Protect From Potential Harmful Radiation” (filed Sep. 30, 2021), the entirety of which foregoing application is incorporated herein by reference for any and all purposes.

### TECHNICAL FIELD

The present disclosure relates to the field of MXene materials and to the field of ophthalmic lenses.

### BACKGROUND

Color vision deficiency (CVD) sufferers experience difficulties in daily life, as accuracy in color differentiation plays an essential role. Implications of the disorder limit career prospects, such as in the armed forces, where basic requirements cannot be met.

Current methods to manage CVDs include, e.g., patient specific color filtering, spectacles, and contact lenses. A range of approaches have attempted the challenge to restore normal color vision, in which challenge one much address color filtration while retaining maximum optical transmission.

In many cases, patients desire spectacle independence, which desire has spurred research into CVD contact lens designs. The performance, cost, and scalability of existing approaches, however, limit their utility. Accordingly, there is a long-felt need in the field for improved lens designs useful to those who suffer from CVD.

### SUMMARY

This disclosure provides, inter alia, the incorporation of MXenes with contact/intraocular lens materials in order to target and filter unwanted wavelengths of incoming light with the capability to treat ocular disorders and protect the ophthalmic environment from harmful radiation.

Others have described using materials in contact lenses to filter wavelengths 540-580 nm, and therefore are suggested to treat color vision deficiencies (CVDs), specifically in the red-green region. CVDs effect the ability to identify and differentiate between certain colors.

To date, there are no cures for CVDs, only management of the disorder. Photoreceptor cones forming part of the retina allow colors to be perceived. There are 3 types of cones, short, middle, and long, which correspond to different wavelengths of light. CVDs occur when a cone is missing or defective resulting in distorted information supplied to the brain. When a cone is missing or defective the condition is termed dichromacy and while one approach using nanoparticles suggested a possible treatment for red-green CVD, the most common form, this disclosure establishes that MXenes can filter a wider range of wavelengths.

MXenes are a 2D class of materials that have shown excellent performance in a wide variety of technologies, including energy storage. In 2017, the first demonstration of Ti₃C₂Tₓ cytocompatibility introduced MXenes for biomedical applications. MXenes' tunable, optical, electrochemical and electronic properties can depend not only on the MXene composition but also on flake size and atomic structure. These properties allow this family of 2D materials to be tailored to a broad range of applications. Moreover, their inherent mechanical properties of 2D materials allow their integration within wearable devices when flexibility is required.

MXenes, such as, but not limited to, Ti₂CTₓ and Mo₂TiC₂Tₓ, can have active plasmon peaks of λₘₐₓ=542 nm and λₘₐₓ=476 nm, respectively. These peaks have the potential to filter wavelength bands of 520-580 nm or 440-500 nm, significant color crossovers in the management of CVDs. Additionally, due to MXenes' ability to absorb ultraviolet light, these materials can provide the added benefit of protection against potential harmful radiation.

In one aspect, the present disclosure provides a wearable ophthalmic component, comprising: a matrix material, the matrix material optionally being transparent to visible light; and a MXene material dispersed on and/or in the matrix material, the MXene material being selected and being present at a loading level sufficient to filter light of a visible color.

Also provided are methods, comprising fabricating a component according to the present disclosure (e.g., any one of Aspects 1-10).

Further provided are methods, comprising the use of a component according to the present disclosure (e.g. any one of Aspects 1-10) by an individual experiencing color vision deficiency. It should be understood that a component according to the present disclosure can be configured (e.g., via one or more of choice of MXene material, loading level of MXene material, choice of matrix material) for a particular individual's needs. For example, a component can be configured to filter light in a particular color crossover range (e.g., from 440-500 nm or from 520-580 nm) that is suited to that individual.

Also provided is a method, comprising contacting a MXene and a matrix material to form a color-filtering composition and forming the color-active composition into an ophthalmic component.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various aspects discussed in the present document. In the drawings:

FIG. 1 provides an illustration of the UV-Vis-NIR optical extinction properties of MXenes.

FIG. 2 illustrates the use of a MXene-based contact lens to filter wavelengths in the color crossovers for management of color vision deficiencies (CVD). Schematic representing the mechanism for CVD management.

FIGS. 3A-3C illustrate X-ray diffraction of (FIG. 3A) Ti₃AlC₂ and Ti₃C₂Tₓ, (FIG. 3B) Ti₂AlC and Ti₂CTₓ and (FIG. 3C) Mo₂TiAlC₂ and Mo₂TiC₂Tₓ.

FIG. 4 illustrates X-ray diffraction of PVA hydrogel, Ti₃C₂Tₓ/PVA hydrogel, Ti₂CTₓ/PVA hydrogel and Mo₂TiC₂Tₓ/PVA hydrogel.

FIGS. 5A-5B provide SEM images of a freeze-dried PVA hydrogel EHT=1 kV at 125× magnification; FIG. 5A illustrates a hydrogel and FIG. 5B illustrates the hydrogel of FIG. 5B in swollen form. Optical images were made using the laser mode of Keyence Microscope, 20× magnification.

FIGS. 6A-6C provide (FIG. 6A) an optical photograph of PVA hydrogels with varying MXene wt %, (FIG. 6B) extinction spectra of UV-vis region of MXene/PVA hydrogels, and (FIG. 6C) Calibration curve at 770 nm (lambda max) at varying Ti₃C₂Tₓ/PVA ratios.

FIGS. 7A-7C provide (FIG. 7A) a photograph of Mo2TiC2Tx/PVA hydrogels with increasing Mo2TiC2Tx content (starting with 0 wt %). UV-vis spectra of (FIG. 7b) Mo2TiC2Tx/and (FIG. 7C) Ti2CTx/PVA hydrogels with increasing MXene content.

FIGS. 8A-8B provide (FIG. 8A) Effect of increasing hydrogel thickness (1.5, 2.3, 3.8 and 7.7 mm) and MXene content (HG-1<HG-4), in transmittance at relevant wavelength (476 nm), and (FIG. 8B) photograph of hydrogels of different thicknesses and MXene content.

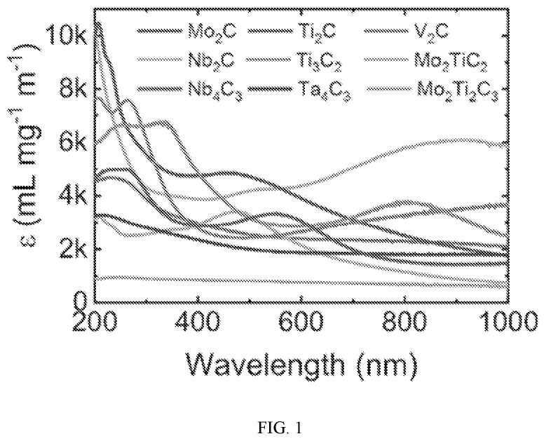

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints (e.g., “between 2 grams and 10 grams, and all the intermediate values includes 2 grams, 10 grams, and all intermediate values”). The endpoints of the ranges and any values disclosed herein are not limited to the precise range or value; they are sufficiently imprecise to include values approximating these ranges and/or values. All ranges are combinable.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. In at least some instances, the approximating language may correspond to the precision of an instrument for measuring the value. The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4. Further, the term “comprising” should be understood as having its open-ended meaning of “including,” but the term also includes the closed meaning of the term “consisting.” For example, a composition that comprises components A and B can be a composition that includes A, B, and other components, but can also be a composition that includes only A and B. Any documents cited herein are incorporated by reference in their entireties for any and all purposes.

The human eye is responsible for the detection and interpretation of light from the environment where sight and perception are reliant on the correct functionality of its components. Up to 8% of males and 0.5% of females suffer from colour vision deficiencies (CVDs), which are visual disorders that affect the ability to differentiate between certain colors¹.

Rod and cone photoreceptors, forming the retina, are responsible for the collection of light. Rods are accountable for vision in low light conditions, while cones are capable of interpreting a wide spectrum of photons responsible for colour vision². Cones differ in length and correspond to specific colours, short(S), middle (M) and long (L) corresponding to blue, green and red respectively.

Trichromatism is used to describe normal colour vision where all three types of S-, M-, and L-cones work together to perceive all hues of colour. CVDs occur when a cone is missing or defective resulting in misinformation supplied to the brain, termed dichromacy^{2 3 4}. Dichromacy can be categorised as deuteranomaly, affecting red-green vision, the most common type of CVD, protanomaly, where reds appear less bright, and tritanomaly, the least common type of CVD that makes it difficult to differentiate between blue and green and between yellow and red.

The occurrence of CVDs vary from mild to more severe, as a result of physical damage or most commonly, congenitally through a recessive trait on the X chromosome⁵. There are no current cures for people with CVDs, only management of the disorder. Accordingly, people living with CVDs experience difficulties completing simple daily activities. Moreover, people are limited with regards to career options when basic requirements are unable to be met.

Whilst CVD awareness has seen a significant increase, as research towards gene therapy appears promising, it remains in its infancy. Another approach, used in the management of the disorder, is tinted spectacles. Such devices can utilise optical lens technology by filtering wavelengths of light through optical fibres. Missing or defective cones result in misinformation transferred to the brain, the filtration of a range of wavelengths aims to increase clarity of interpretation of the S-, M-, and L-cones. With spectacle independence the goal for many, research interests have turned to developments in contact lenses as a novel approach to wavelength filtration.

Several approaches have been explored to address wavelength filtration in contact lenses for the purpose of CVD management. One approach related to using dyed contact lenses to filter wavelengths of 545-575 nm through the incorporation of a rhodamine derivative to the base-polymer. The lens demonstrated slight improvements in colour perception whilst experiencing no toxicity to corneal epithelial cells. Although colour vison comparable to normal colour vision was not achieved.

In another study, turmeric, spinach, paprika, and woad powders were incorporated into contact lenses to investigate their individual wavelength filtering capabilities. Turmeric showed potential in filtering ultra-violet radiation that could potentially damage the retina. Whereas spinach, paprika, and woad powder loaded polymers were found to mitigate >20% of visible light transmission. Another investigation explored the use of gold nanoparticles (GNP) to treat red-green CVD. A gold nanocomposite (GNC) was fabricated with optical and material properties comparable to commercial and research-based CVD treatments. Even though their work demonstrates the potential of nanoparticles as selective light filters, the cost and scalability limit the commercialization inviting the exploration of other nanomaterials.

MXenes' composition-dependent optical properties can be defined, demonstrating their active plasmon peaks spanning over the ultraviolet-visible-Near infrared (UV-Vis-NIR) region. Ti₂CTₓ or Mo₂TiC₂Tₓ exhibited extinction peaks of λₘₐₓ=542 nm and λₘₐₓ=476 nm (FIG. 33), showing capability to block wavelengths 520-580 nm and 440-500 nm which are relevant colour crossovers in the management of CVDs.

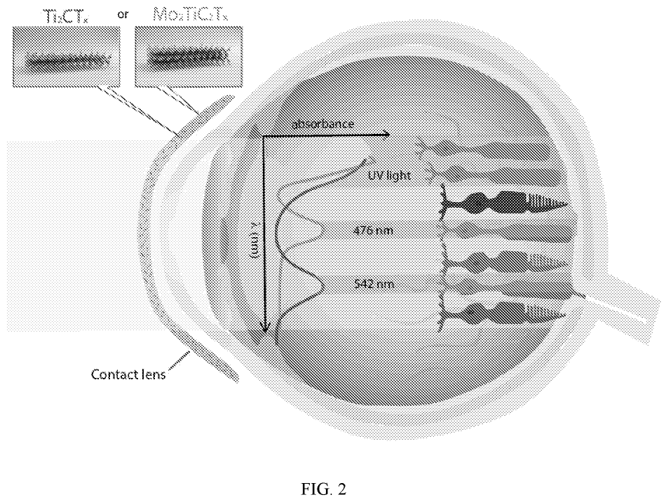

Consequently, by selecting a suitable MXene composition, the ophthalmic device can be tuned appropriately for a patient's specific requirements. In connection with this discovery, we present here the incorporation of MXenes in wearable or implantable ophthalmic devices such as glasses, contact lenses and intraocular lenses to selectively filter light of certain wavelengths, which filtration can allow CVD patients to overcome everyday challenges (FIG. 2). Furthermore, due to the ability of MXenes to absorb ultraviolet light, these offer the added benefit of simultaneously protecting the eye from harmful radiation. In the development of multifunctional lenses for CVD correction and UV protection, one can further the application of MXenes beyond Ti₃C₂Tₓ in wearable ocular devices, and the fabrication of MXene-based lenses is cost-effective and scalable. Thus, the present disclosure provides components that can be used for CVD correction and/or for UV protection, including components that are useful in both applications.

Here we demonstrate the use of Ti₂CTₓ and Mo₂TiC₂Tₓ (as example, non-limiting MXenes) to offer wavelength blocking capabilities for CVD management. The example MXenes listed were synthesised and physical characterization performed. The MXenes were incorporated during the synthesis of polyvinyl alcohol (PVA) hydrogels to exemplify their light filtering performance and their respective stability was evaluated.

### MXenes

A MXene composition is, generally, any of the compositions described in at least one of U.S. patent application Ser. No. 14/094,966 (filed Dec. 3, 2013), 62/055,155 (filed Sep. 25, 2014), 62/214,380 (filed Sep. 4, 2015), 62/149,890 (filed Apr. 20, 2015), 62/127,907 (filed Mar. 4, 2015) or International Applications PCTx/US2012/043273 (filed Jun. 20, 2012), PCTx/US2013/072733 (filed Dec. 3, 2013), PCTx/US2015/051588 (filed Sep. 23, 2015), PCTx/US2016/020216 (filed Mar. 1, 2016), or PCTx/US2016/028,354 (filed Apr. 20, 2016), preferably where the MXene composition comprises titanium and carbon (e.g., Ti₃C₂, Ti₂C, Mo₂TiC₂, etc.). Additional MXenes are also described in PCTx/US2020/054912 (filed Oct. 9, 2020). Any one or more of the compositions described in the foregoing applications can be used in the presently-disclosed technology.

Although MXene compositions include any and all of the compositions described in the patent applications and issued patents mentioned elsewhere herein, in some embodiments, MXenes can be materials comprising or consisting essentially of a Mₙ₊₁Xₙ(Tₛ) composition having at least one layer, each layer having a first and second surface, each layer comprising:
a substantially two-dimensional array of crystal cells.
each crystal cell having an empirical formula of Mₙ₊₁Xₙ, such that each X is positioned within an octahedral array of M,
wherein M is at least one Group 3, 4, 5, 6, or 7,
wherein each X is C and/or N, and
n=4;
wherein at least one of said surfaces of the layers has surface terminations, Tₛ, independently comprising alkoxide, alkyl, carboxylate, halide, hydroxide, hydride, oxide, sub-oxide, nitride, sub-nitride, sulfide, sulfonate, thiol, or a combination thereof.

As described elsewhere within this disclosure, the Mₙ₊₁Xₙ(Tₛ) materials produced in these methods and compositions have at least one layer, and sometimes a plurality of layers, each layer having a first and second surface, each layer comprising a substantially two-dimensional array of crystal cells; each crystal cell having an empirical formula of Mₙ₊₁Xₙ, such that each X is positioned within an array of M, wherein M is at least one Group 3, 4, 5, 6, or 7 metal (corresponding to Group IIIB, IVB, VB, VIB or VIIB metal), wherein X is C and/or N and n=4; wherein at least one of said surfaces of the layers has surface terminations, Tₛ, comprising alkoxide, alkyl, carboxylate, halide, hydroxide, hydride, oxide, sub-oxide, nitride, sub-nitride, sulfide, sulfonate, thiol, or a combination thereof.

Supplementing the descriptions above, Mₙ₊₁Xₙ(Tₛ), compositions may be viewed as comprising free standing and stacked assemblies of two dimensional crystalline solids. Collectively, such compositions are referred to herein as “Mₙ₊₁Xₙ(Tₛ),” “MXene,” “MXene compositions,” or “MXene materials.” Additionally, these terms “Mₙ₊₁Xₙ(Tₛ),” “MXene,” “MXene compositions,” or “MXene materials” also refer to those compositions derived by the chemical exfoliation of MAX phase materials, whether these compositions are present as free-standing 2-dimensional or stacked assemblies (as described further below). Reference to the carbide equivalent to these terms reflects the fact that X is carbon, C, in the lattice. Such compositions comprise at least one layer having first and second surfaces, each layer comprising: a substantially two-dimensional array of crystal cells; each crystal cell having an empirical formula of Mₙ₊₁Xₙ, where M, X, and n are defined above. These compositions may be comprised of individual or a plurality of such layers. In some embodiments, the Mₙ₊₁Xₙ(Tₛ) MXenes comprising stacked assemblies may be capable of, or have atoms, ions, or molecules, that are intercalated between at least some of the layers. In other embodiments, these atoms or ions are lithium. In still other embodiments, these structures are part of an energy-storing device, such as a battery or supercapacitor. In still other embodiments these structures are added to polymers to make polymer composites.

The term “crystalline compositions comprising at least one layer having first and second surfaces, each layer comprising a substantially two-dimensional array of crystal cells” refers to the unique character of these materials. For purposes of visualization, the two-dimensional array of crystal cells may be viewed as an array of cells extending in an x-y plane, with the z-axis defining the thickness of the composition, without any restrictions as to the absolute orientation of that plane or axes. It is preferred that the at least one layer having first and second surfaces contain but a single two-dimensional array of crystal cells (that is, the z-dimension is defined by the dimension of approximately one crystal cell), such that the planar surfaces of said cell array defines the surface of the layer; it should be appreciated that real compositions may contain portions having more than single crystal cell thicknesses.

That is, as used herein, “a substantially two-dimensional array of crystal cells” refers to an array which preferably includes a lateral (in x-y dimension) array of crystals having a thickness of a single cell, such that the top and bottom surfaces of the array are available for chemical modification.

Metals of Group 3, 4, 5, 6, or 7 (corresponding to Group IIIB, IVB, VB, VIB, or VIIB), either alone or in combination, said members including, e.g., Ti, Zr, Hf, V, Nb, Ta, Cr, Mo, and W. For the purposes of this disclosure, the terms “M”, or “M′”, or “M″”, or “M atoms,” “M elements,” or “M metals” may also include Mn. Also, for purposes of this disclosure, compositions where M comprises Ti, Zr, Hf, V, Nb, Ta, Cr, Mo, W, or mixtures thereof constitute independent embodiments. Similarly, the oxides of M may comprise any one or more of these materials as separate embodiments. For example, M may comprise any one or combination of Hf, Cr, Mn, Mo, Nb, Sc Ta, Ti, V, W, or Zr. In other preferred embodiments, the transition metal is one or more of Ti, Zr, V, Cr, Mo, Nb, Ta, or a combination thereof. In even more preferred embodiments, the transition metal is Ti, Ta, Mo, Nb, V, Cr, or a combination thereof.

The range of compositions available can be seen as extending even further when one considers that each M-atom position within the overall Mₙ₊₁Xₙ matrix can be represented by more than one element. That is, one or more type of M-atom can occupy each M-position within the respective matrices. In certain exemplary non-limiting examples, these can be (M′ₐM″_{b})X₄, where M′ and M″ are different metals (e.g., members of the same group), and a+b=5; or (M′ₐM″_{b})₅X₄Tₓ, where M′ and M″ are different metals (e.g., members of the same group), and a+b=1. As some non-limiting examples, such a composition can be (V_{1/2}Nb_{1/2})₅C₄ or (V_{1/3}Nb_{2/3})₅N₄.

In the same way, one or more type of X-atom can occupy each X-position within the matrices, for example solid solutions of the formulae M₅(CⱼNₖ)₄ (where j+k=1); (M′ₐM″_{b})(CⱼNₖ)₄ (where a+b=5 and j+k=1); and (M′ₐM″_{b})₅(CⱼNₖ)₄ (where a+b=1 and j+k=1).

### Example Disclosure

It should be understood that the MXenes, substrates, and other materials mentioned herein are illustrative only and do not limit the scope of the present disclosure or the appended claims. As an example, the use of Ti₃C₂Tₓ MXenes herein should not be understood as limiting the disclosed technology to using only Ti₃C₂Tₓ MXenes.

### Materials

Hydrofluoric acid (HF), hydrochloric acid (HCl), lithium chloride (LiCl) (Acros Organics, USA), Ti₃AlC₂, Ti₂AlC₂ and Mo₂TiAlC₂ (A.J. Drexel Nanomaterials Institute, Drexel University, USA), polyvinyl alcohol (Aldrich, USA), dimethyl sulfoxide (Acros Organics, USA), deionized (DI) water.

### MXene Synthesis

Ti₃C₂Tₓ(Ti₂CTₓ and Mo₂TiC₂Tₓ) flakes were synthesized from high-aluminium Ti₃AlC₂(Ti₂AlC₂ and Mo₂TiAlC₂) MAX phase using the hydrofluoric-hydrochloric (HF—HCl) etching method¹². The aluminium layer was first chemically etched from the MAX phase. The etchant consisted of a mixture of 12 M HCl, deionized (DI) water and 50 wt % HF in a volume ratio of 6:3:1. Once the etchant was prepared, 1 g of MAX phase was added slowly (1 g/5 mins) to the etchant in a polyethylene bottle. A magnetic stirred was added and the bottle was lightly capped to allow gas release.

The solution was left to stir for 24 hrs (16 and 48 hrs) at 35 degrees Celsius (° C.) at 400 rounds per minute (rpm). The resulting acidic solution consisted of MXene flakes held together by van der Waals bonds, known as multi-layered MXene. The solution was centrifuged for 5 mins at 3,500 rpm obtaining a clear supernatant which was decanted into a waste container and the sediment was redispersed in DI water.

This cycle was repeated until the pH, measured with a pH strip, reached 6. After the last centrifugation run, the supernatant was decanted, and the neutralized sediment was kept for delamination.

For delamination, 1 g of lithium chloride (LiCl) was mixed with 50 mL of DI water. The 0.5 M LiCl solution was used to redisperse the neutralized sediment. The solution was left stirring for 24 hours at 400 rpm. The solution was collected and through a series of centrifugation runs at 3500 rpm with the first lasting only 5 mins and the following lasting 1 hr, delaminated MXene was washed.

The supernatant of the last wash containing single-layered Ti₃C₂Tₓ (Ti₂CTₓ and Mo₂TiC₂Tₓ) was decanted and stored in a closed container in the fridge until use.

### Fabrication of MXene-Based Hydrogels

In a 20 mL glass vial, 1 g of PVA was weighed followed by 6.25 mL of DMSO in DI water with the ratio 8:2 (v/v). The PVA in DMSO was placed into a 70° C. oil bath with constant stirring (400 rpm) for 3 hrs. In a separate vial, 2 mL of the DMSO/DI water 8:2 (v/v) was used to dilute a known volume of MXene. Once the 3 hrs had passed, the 2 mL of MXene/DMSO were pipetted into the glass vial while stirring in the oil bath for 5-10 mins. 1 g of the resulting homogeneous paste was immediately transferred into a 3×3 cm² plate which served as the mould of the hydrogel. Finally, the moulds were placed into a −20° C. freezer and left overnight for polymerization. The moulds were then removed from the freezer and left at room temperature for the hydrogels to thaw.

The hydrogels were placed into a container filled with DI water and shaken in plate shaker for 1 hr. Then, the water was changed for fresh DI water. This cycle was repeated 3 times to exchange the DMSO with DI water. The hydrogels were stored in the lab drawer in closed vials filled with DI water.

By increasing or decreasing the mass of MXene in the 2 mL of DMSO/DI water, the final weight percent of MXene in the hydrogel was controlled.

### Characterization

X-ray diffraction (XRD) patterns (Rigaku Smartlab) were used to characterize MAX phases, MXenes and MXene/PVA hydrogels. For sample preparation, MAX phase powders were texturized, MXenes colloidal solutions were vacuum-filtered and hydrogels were freeze-dried. The PVA hydrogel was also imaged via scanning electron microscopy (SEM, Zeiss Supra 50VP). The measurements were performed at an electron height tension (EHT) of 1 kV in high vacuum mode.

Samples were platinum sputtered. Optical microscopy was used to capture images of the hydrogel using a Keyence microscope (VHK-7000). UV-Vis spectroscopy (Evolution 201, Thermo Scientific, USA) was used to evaluate the absorbance spectra of the hydrogels in quarts slides.

### Results and Discussion

### MXene Synthesis and Characterization

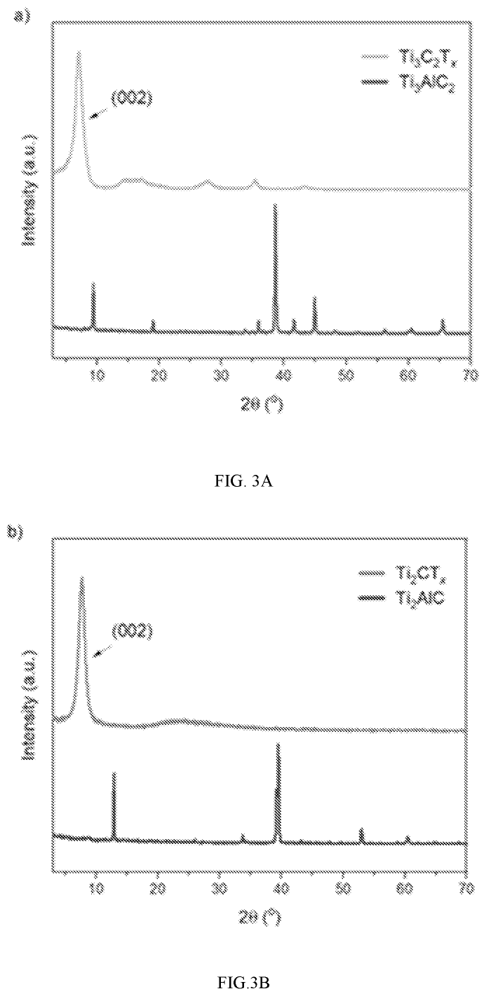

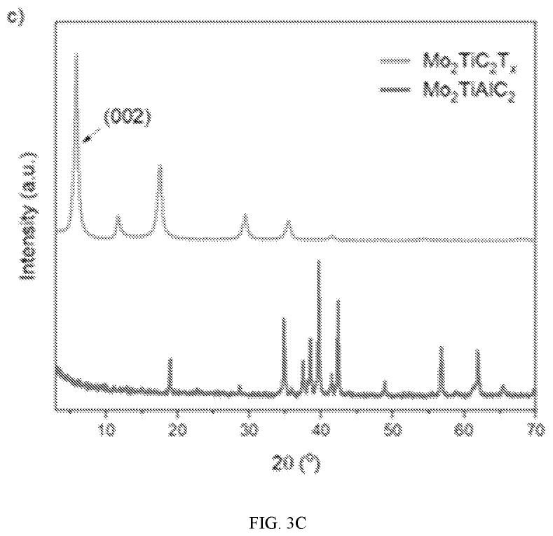

Several methods for synthesis of delaminated Ti₃C₂Tₓ flakes have been reported in the literature. It can be useful to consider the method in view of the ultimate application, considering that the conditions under which MXenes are synthesized influence the properties of the end material, In 2021, researchers at Drexel University proposed an improved synthesis method for the MAX phase precursor, that in turn results in increased stability and conductivity of the resultant MXene product. In short, an excess of aluminium during MAX phase synthesis produces Ti₃AlC₂ with better stoichiometry and crystallinity. Therefore, this precursor synthesis method was selected for use within the project, resulting in higher quality delaminated Ti₃C₂Tₓ. FIG. 3 illustrates the XRD for MAX phases and MXenes, showing a shift of the 002 peak from a higher to a lower peak position (°) after MXene synthesis due to the removal of the Al layers. The 002 peak can be used on Bragg's equation to calculate the interlayer spacing. Therefore, the interlayer spacing increased from 0.94 to 1.25 nm for Ti₃C₂Tₓ, from 0.69 to 1.15 nm for Ti₂CTₓ and from 0.47 to 1.42 nm for Mo₂TiC₂Tₓ.

### MXene/PVA Hydrogel Fabrication and Characterization

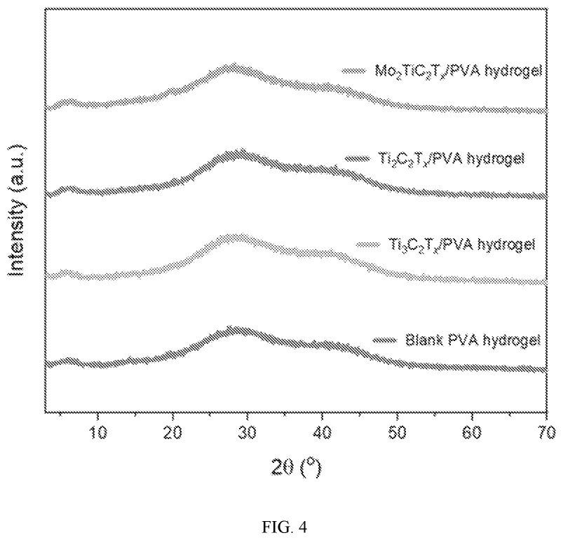

The synthesized MXenes were incorporated to PVA hydrogels during their synthesis. XRD spectra of PVA-hydrogel and MXene-PVA hydrogels are shown in FIG. 4. As expected, all XRD spectra show high noise due to the random polymeric network. Due to the low content (<0.2 wt %) of MXene in each hydrogel, and the random distribution throughout the hydrogel, no characteristic peaks are identified in the XRD. Instead, the UV-vis spectra recorded for each hydrogel will serve as a confirmation of the successful incorporation of MXene to the polymeric network.

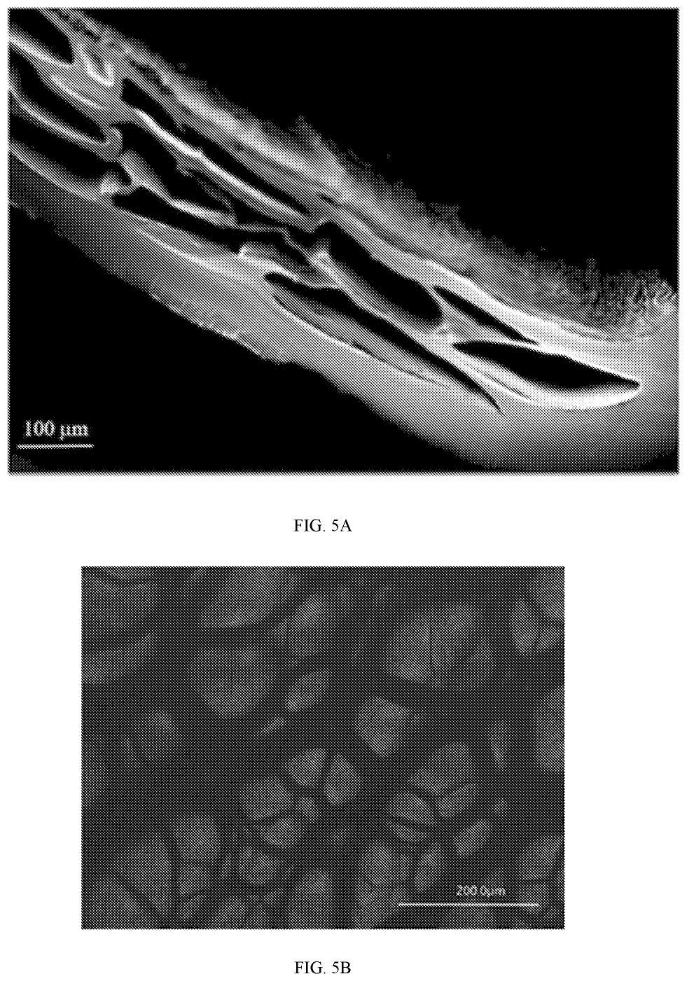

The ability of hydrogels to retain high water content (>80%) is due to their three-dimensional polymeric networks forming macroscopical pores that enable large quantities of water to be stored. SEM was used to capture a cross-sectional image of the Ti₃C₂Tₓ/PVA hydrogel (FIG. 5a). Ti₃C₂Tₓ was initially used for preliminary work to assess hydrogel fabrication, due to its high stability and that it can be dispersed homogeneously in polymer matrixes. The successful fabrication of polymetric networks are observed in FIG. 5A with pore sizes approximately 200 nm. Without being bound to any particular theory or embodiment, the slip-shaped pores in FIG. 5A are attributed to the sample preparation. Hydrogels were freeze-dried in an effort to maintain their structure; during preparation it was noted that the hydrogels collapsed after drying. Furthermore, when cutting the sample for cross-sectional imaging, it is likely that the pressure applied by the razor blade contributed further to structural collapse.

While the SEM image shows a hydrogel thickness of around 0.5 mm, the swollen hydrogels had an approximate thickness of 2 mm. Therefore, in order to assess more accurately the true shape of the pores, optical images were taken of the swollen hydrogels (FIG. 5B). The laser mode of the Keyence microscope enabled imaging of the hydrogels. Circular-shaped pores were observed, agreeing with the pore size of the hydrogels if the structure had not collapsed during sample preparation.

Ti₃C₂Tₓ/PVA Hydrogel Characterisation for Preliminary Study

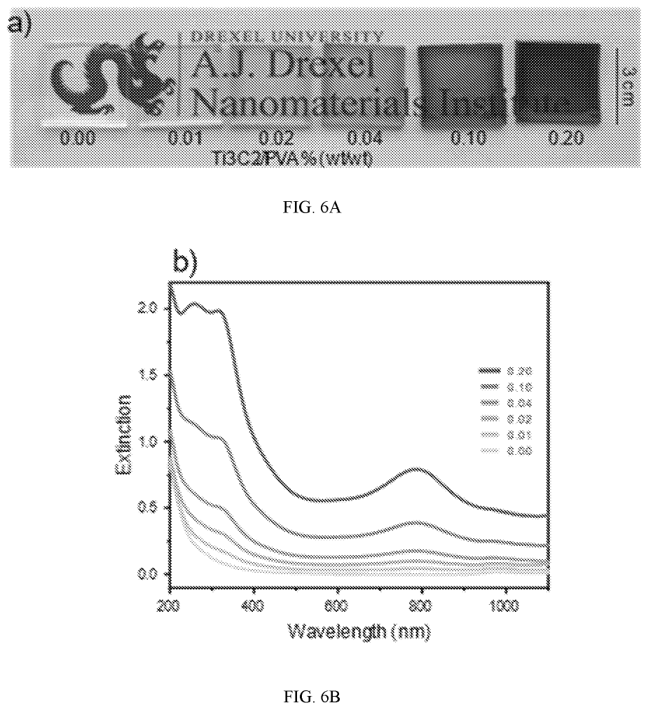

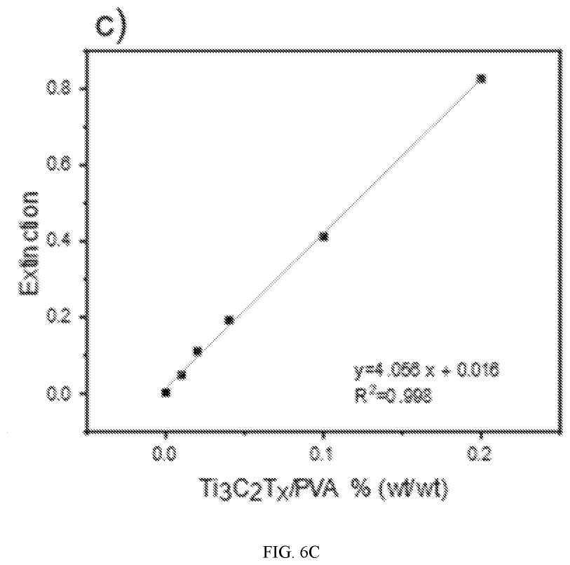

The digital photograph in FIG. 6a shows PVA hydrogels with 0 to 0.20 wt % of Ti₃C₂Tₓ. As a result of the synthesis method the hydrogel without MXene is highly transparent. Holloway et al. optimised the PVA wt % and the solvent ratio between DMSO and DI water to achieve maximum transparency¹⁵. Furthermore, Gupta et al. showed that the number of crystallites, which scatter light, increased with an increase of number of freezing-thawing cycles¹⁶. Therefore, the choice of synthesis was based on these observations. In brief, 10 wt % PVA was polymerised in a 80/20 DMSO to DI water (v/v) solvent with only one freezing-thawing cycle. The observed green colour in the hydrogels is inherent of Ti₃C₂Tₓ.

The UV-vis extinction (absorption and reflectance) spectra of MXene hydrogels with different MXene content are shown in FIG. 6a. Since Ti₃C₂Tₓ has a characteristic peak around 770 nm in the visible region, a peak at the same wavelength was expected on those samples containing Ti₃C₂Tₓ. This peak was assigned to be the plasmon resonance elsewhere using electron energy loss spectroscopy (EELS). As expected, the hydrogel with no MXene content lacks the resonance peak characteristic of Ti₃C₂Tₓ. Furthermore, the low extinction throughout the spectra for the hydrogel without MXene confirms that the hydrogel is highly transparent as PVA is silent in the UV-vis region. Inspired by the Beer-Lambert Law (Eq. 1), which relates the material concentration to absorption linearly by absorbidity (epsilon), the hydrogels extinction (or absorption, A) at lambda=780 nm, was plotted against the MXene content. As shown in FIG. 6b, this follows a linear trend suggesting that UV-vis spectroscopy can also be used to determine the MXene content (MXene concentration, 6c) within the hydrogel as long as the rest of the parameters remain such as hydrogel thickness (path length, l), which are discussed elsewhere herein.

$$A = ε c λ \qquad ( Eq . 1 )$$

Mo₂TiC₂ and Ti₂C Performance for CVD Management

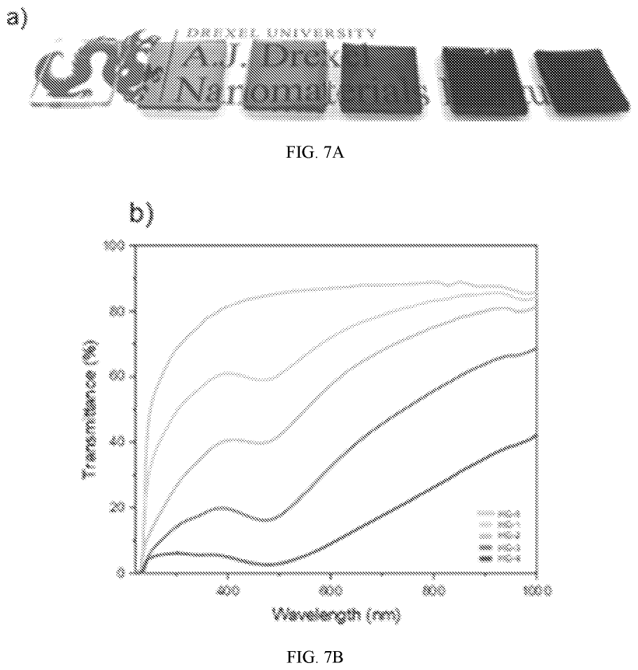

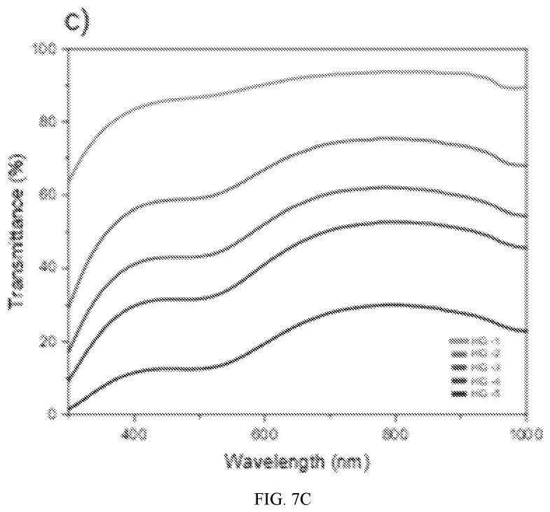

The next step was to test the performance of Mo₂TiC₂Tₓ- and Ti₂CTₓ-PVA hydrogels to block the wavelength of light relevant for CVD. FIG. 7a, shows a photograph of the Mo₂TiC₂Tₓ-PVA hydrogels with increasing concentrations of Mo₂TiC₂Tₓ. Contrastingly to Ti₃C₂Tₓ-PVA hydrogels, these hydrogels take a brownish colour which is characteristic from Mo₂TiC₂Tₓ due to the location of its plasmonic peak.

From left to right, with increasing Mo₂TiC₂Tₓ content, the transparency of the hydrogels decreases (FIG. 7a). Hydrogels were named with increasing numbering to denote a relative increase in MXene content. Transmittance, which refers to the amount of light that can pass through the material, of the hydrogels was recorded throughout the UV-vis spectra using a UV-vis spectroscopy. It is observed that with increasing MXene content, the transmittance decreases as expected. Moreover, a dip is observed around λ=476 nm and λ=530 nm, for Mo₂TiC₂Tₓ- and Ti₂CTₓ-PVA hydrogels, respectively. This decrease in transmittance at these wavelengths may be attributed to the plasmonic resonance similar to what is observed for Ti₃C₂Tₓ. These results validate the use of MXenes for colour vision deficiencies, specifically Mo₂TiC₂Tₓ for management of deficiencies in the red-green crossover and Ti₂CTₓ for blue-green crossover.

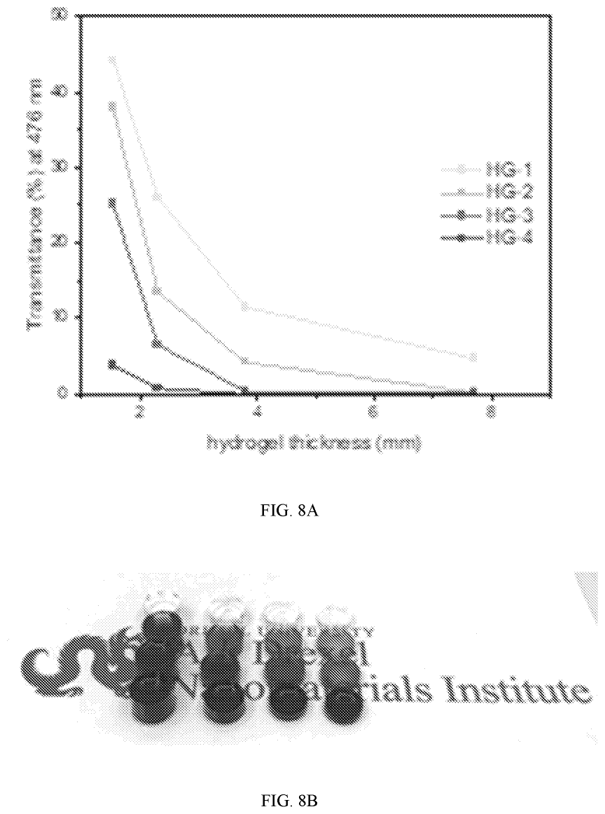

It has been demonstrated that the Mo₂TiC₂Tₓ and Ti₂CTₓ can block light at the relevant wavelengths for CV management. Furthermore, it is observed that the transmittance of light can be tailored by increasing or decreasing the MXene content in the hydrogel. In FIG. 8, the effect of the hydrogel thickness is also analysed and exemplified with Mo₂TiC₂Tₓ/PVA hydrogels. FIG. 8a, is a plot of the transmittance at λ=476 nm against the relative thickness of the hydrogel. To vary the hydrogel thicknesses (1.5, 2.3, 3.8 and 7.7 mm), different amounts of MXene/PVA homogenous solution were added to the same size mould resulting in changes of dimensions in the z-direction as shown in FIG. 8b. It was observed that with an increase of hydrogel thickness the transmittance is decreased. Those hydrogels with higher content of Mo₂TiC₂Tₓ (HG-3 and HG-4), block most of the light even at the thinner thickness (1.5 and 2.3 mm). No quantitative analysis was performed, considering that the hydrogel thickness was controlled by polymer weight added to the mould during synthesis and the thickness measurement was done manually without high precision tools. However, the observed trends of decreasing transmittance with increasing thickness and/or increasing MXene content allows the tailoring of MXene-based hydrogels for contact lenses for CVD management.

A stability study of the hydrogels was performed by recording the UV-vis spectra of Mo₂TiC₂Tₓ and Ti₂CTₓ-PVA hydrogels periodically. The dip in transmittance was used to monitor stability of the hydrogel which consequently determines the lifetime of the hydrogels for potential use of contact lenses for CVD. Considering that the plasmonic peak is characteristic of the MXene under study, when MXene degrades into a different material, this peak decreases. The dip in transmittance for the Mo₂TiC₂Tₓ-PVA hydrogel remains stable after more than 1 month of storing the hydrogel on the laboratory bench top at room temperature in DI water. Whereas, the plasmonic peak of Ti₂CTₓ-PVA hydrogel disappears after 3 days. Both results are in accordance with the stability of their respective MXenes when stored as colloidal solutions in DI water.

To lengthen the lifetime of Ti₂CTₓ-PVA hydrogels, one can store hydrogels in the freezer. It is well known that the rate of oxidation resulting from its interaction with water decreases at lower temperatures. By modulating the storage conditions, as it has been done for MXene colloidal solutions, one can increase the lifetime of MXene-based hydrogels.

### CONCLUSIONS

We have demonstrated the potential of MXenes for the application of CVD management. A simple synthesis approach for MXene/PVA hydrogels was introduced to achieve high transparency hydrogels which is of foremost importance for CLs. MXenes were successfully incorporated to hydrogels during polymerization confirmed by their corresponding UV-vis spectra. The effect of MXene content and hydrogel thickness were explored to enable tailoring transmittance at the wavelength relevant to the specific type of CVD deficiency. Furthermore, the stability study shows that the fabricated Mo₂TiC₂Tₓ/PVA hydrogels correspond with the shelf-life of long-term CLs currently on the market. For Ti₂CTₓ-PVA hydrogels, the shelf-life is approximately 2 days when stored at room temperature which is sufficient for 1-day wear CLs. In addition to using MXenes in CLs, MXenes can be used coatings on spectacles (or even incorporate into lenses) as an alternative way to manage CVD. Without being bound to any particular theory, the shelf-life of MXene-based glasses may be especially strong, as oxygen dissolved in water is the main contributor for MXene degradation.

It should be understood that although hydrogels were used as example matrix materials, the present disclosure is not limited to the use of hydrogels (which includes silicone hydrogels) as the matrix material. Other materials (e.g., non-hydrogel polymers and even glass) can also be used. For example, the disclosed technology can be used to form safety glasses (e.g., UV-protective and/or CVD-treating safety glasses used by workers in sunny environments), which safety glasses can comprise a MXene and a non-hydrogel polymer, e.g., polycarbonate, PMMA, and the like. A hydrogel matrix can be used for a component that contacts the eye (e.g., a contact lens), and a non-hydrogel matrix can be used for a component that does not contact the user's eye, e.g., glasses, face shields, and visors.

### Aspects

The following Aspects are illustrative only and do not limit the scope of the present disclosure or the appended claims. Any part of any Aspect can be combined with any part or any parts of any other Aspect.

Aspect 1. A wearable ophthalmic component, comprising: a matrix material, the matrix material optionally being transparent to visible light; and a MXene material dispersed in and/or on the matrix material, the MXene material being selected and being present at a loading level sufficient to filter light of a visible color.

Such a component can be, e.g., a lens (such as a contact lens or a glasses lens). The component can be a visor, face shield, or other wearable component.

The matrix material can be transparent to visible light, but this is not a requirement. The matrix material can be tinted or otherwise colored, independent of the presence of any MXene.

A matrix material can comprise, e.g., a polymeric material, such as a hydrogel. The wearable component can be configured as a lens, e.g., as a corrective lens that corrects a vision deficiency (e.g., near-sightedness, far-sightedness, astigmatism) of a user. As described elsewhere herein, a lens can be an eyeglass lens and/or a contact lens. The lens can be an exterior or external lens (e.g., a contact lens, an eyeglass lens), as opposed to an intraocular lens (IOL).

The MXene material can be, e.g., dispersed within the matrix material. Such dispersion can be, e.g., an intimate mixture between the MXene and the matrix material. The MXene material can also be disposed on the matrix material, e.g., disposed on or within pores or other void spaces of the matrix material. In some embodiments, the MXene can be present as a coating on the matrix material, e.g., as a spin coating. A MXene can be taken up by the pores of a porous matrix material, e.g., a porous lens material.

Aspect 2. The component of Aspect 1, wherein the component selectively filters light having a wavelength of from about 440 to about 500 nm. A component can be configured so as to selectively filter light in the ultraviolet range, i.e., from about 10 or about 100 nm to about 500 nm, including one or more of UV-A (315-500 nm), UV-B (280-315 nm), and/or UV-C (100-280 nm), and subranges and combinations thereof. The filtration can be accomplished by, e.g., selection of a particular MXene or MXenes, loading of a particular MXene or MXenes, or both. The MXene or MXenes can be selected to selectively filter light having a wavelength of, e.g., about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, or even about 600 nm.

Aspect 3. The component of Aspect 2, wherein the component selectively filters light having a wavelength of about 476 nm.

Aspect 4. The component of Aspect 1, wherein the component selectively filters light having a wavelength of from about 520 to about 580 nm.

Aspect 5. The component of Aspect 4, wherein the component selectively filters light having a wavelength of about 530 nm.

Aspect 6. The component of any one of Aspects 1-5, wherein the matrix comprises a hydrogel.

Aspect 7. The component of any one of Aspects 1-6, wherein the component is characterized as a contact lens.

Aspect 8. The component of any one of Aspects 1-6, wherein the component is characterized as an eyeglass lens. Such a lens can comprise MXene material disposed within the matrix (e.g., plastic) of the lens. Alternatively, such a lens can comprise a MXene-containing coating disposed on the lens.

Aspect 9. The component of any one of Aspects 1-8, wherein the MXene material comprises at least one of Mo₂TiC₂Tₓ, Ti₃C₂Tₓ, and Ti₂CTₓ.

Aspect 10. The component of any one of Aspects 1-9, wherein the MXene material is present at up to about 2 wt % or 1 wt % of the matrix material. The MXene material can be present at, e.g., from about 0.0001 to about 1 wt % of the matrix material, from about 0.001 to about 0.80 wt %, from about 0.005 to about 0.65 wt %, from about 0.01 to about 0.50 wt %, from about 0.05 to about 0.38 wt %, from about 0.073 to about 0.33 wt %, from about 0.09 to about 0.27 wt %, from about 0.13 to about 0.22 wt %, or even from about 0.14 to about 0.20 wt % of the total weight of the matrix material. As described elsewhere herein, a component can include a single MXene or two or more MXenes. For example, a component can include both Ti₃C₂Tₓ and Ti₂CTₓ.

Aspect 11. A method, comprising fabricating a component according to any one of Aspects 1-10. Without being bound to any particular theory or embodiment, a component can be fabricated according to a particular user's needs, e.g., a component that is configured to selectively filter light having a wavelength of about 530 nm. Such a fabrication can be in accordance with a set of instructions, e.g., instructions developed in connection with a patient's physical/ophthalmic examination. The selection of a MXene or MXenes can be in accordance with a library that maps the filtration capabilities of different MXenes to particular wavelengths of light so as to allow a user to select the MXene or MXenes that are most suited to the particular need of a user, e.g., a need to filter illumination at about 530 nm.

Aspect 12. A method, comprising the use of a component according to any one of Aspects 1-10 by an individual experiencing a color vision deficiency.

Aspect 13. A method, comprising the use of a component according to any one of Aspects 1-10 by an individual in need of protection from ultraviolet radiation.

Aspect 14. A method, comprising contacting a MXene and a matrix material to form a color-filtering composition and forming the color-active composition into an ophthalmic component. Such a component can be a component according to the present disclosure, e.g., according to any one of Aspects 1-10.

Aspect 15. The method of Aspect 14, wherein the matrix material is porous.

Aspect 16. The method of Aspect 14, wherein the matrix material comprises a hydrogel.

Aspect 17. The method of Aspect 14, wherein the MXene is present at up to about 2 wt % of the matrix material in the composition.

Aspect 18. The method of Aspect 17, wherein the MXene is present at up to about 1 wt % of the matrix material in the composition.

Aspect 19. The method of Aspect 17, wherein the ophthalmic component is a contact lens.

Aspect 20. The method of Aspect 17, wherein the ophthalmic component is a glasses lens, a visor, or a faceshield.

### REFERENCES

The following references are included for convenience only. The inclusion of a reference herein should not be understood as an acknowledgment that the reference is material to the patentability of the disclosed technology.
1. Raynor, N. J., Hallam, G., Hynes, N. K. & Molloy, B. T. Blind to the risk: an analysis into the guidance offered to doctors and medical students with colour vision deficiency. Eye 33, 1877-1883 (2019).
2. Neitz, J. & Neitz, M. The genetics of normal and defective color vision. Bone 51, 633-651 (2011).
3. Pastilha, R. C. et al. The colors of natural scenes benefit dichromats. Vision Res. 158, 40-48 (2019).
4. Álvaro, L., Moreira, H., Lillo, J. & Franklin, A. Color preference in red-green dichromats. Proc. Natl. Acad. Sci. U.S.A. 112, 9316-9321 (2015).
5. Aziz, M. Z., Uddin, M. M. & Farooque, U. Prevalence of Color Vision Deficiency among Medical Students in KFU-SA (2014. Int. J. Sci. Res. 5, 53-56 (2016).
6. Gómez-Robledo, L., Valero, E. M., Huertas, R., Martinez-Domingo, M. A. & Hernández-Andrés, J. Do EnChroma glasses improve color vision for colorblind subjects? Opt. Express 26, 28693 (2018).
7. Bastien, K., Mallet, D. & Saint-Amour, D. Characterizing the Effects of Enchroma Glasses on Color Discrimination. Optom. Vis. Sci. 97, 903-910 (2020).
8. Elsherif, M., Salih, A. E., Yetisen, A. K. & Butt, H. Contact Lenses for Color Vision Deficiency. Adv. Mater. Technol. 6, 1-9 (2021).
9. Badawy, A. R. et al. Contact Lenses for Color Blindness. Adv. Healthc. Mater. 7, 1-7 (2018).
10. Sekar, P., Dixon, P. J. & Chauhan, A. Pigmented contact lenses for managing ocular disorders. Int. J. Pharm. 555, 184-197 (2019).
11. Ward, E. J. et al. 2D Titanium Carbide (Ti3C2Tx) in Accommodating Intraocular Lens Design. (2020) doi: 10.1002/adfm.202000841.
12. Anasori, B., Lukatskaya, M. R. & Gogotsi, Y. 2D metal carbides and nitrides (MXenes) for energy storage. Nat. Rev. Mater. 2, (2017).
13. Alexey, L. et al. Elastic properties of 2D Ti3C2Tx MXene monolayers and bilayers. Sci. Adv. 4, eaat0491 (2018).
14. Maleski, K., Shuck, C. E., Fafarman, A. T. & Gogotsi, Y. The Broad Chromatic Range of Two-Dimensional Transition Metal Carbides. 2001563, (2020).
15. Holloway, J. L., Lowman, A. M. & Palmese, G. R. The role of crystallization and phase separation in the formation of physically cross-linked PVA hydrogels. Soft Matter 9, 826-833 (2013).
16. Gupta, S., Goswami, S. & Sinha, A. A combined effect of freeze-thaw cycles and polymer concentration on the structure and mechanical properties of transparent PVA gels. Biomed. Mater. 7, (2012).
17. El-Demellawi, J. K., Lopatin, S., Yin, J., Mohammed, O. F. & Alshareef, H. N. Tunable Multipolar Surface Plasmons in 2D Ti3C2 Tx MXene Flakes. ACS Nano 12, 8485-8493 (2018).

1. A wearable ophthalmic component, comprising
a matrix material,
the matrix material optionally being transparent to visible light; and
a MXene material dispersed in and/or on the matrix material,
the MXene material being selected and being present at a loading level sufficient to filter light of a visible color.

2. The component of claim 1, wherein the component selectively filters light having a wavelength of from about 440 to about 500 nm.

3. The component of claim 2, wherein the component selectively filters light having a wavelength of about 476 nm.

4. The component of claim 1, wherein the component selectively filters light having a wavelength of from about 520 to about 580 nm.

5. The component of claim 4, wherein the component selectively filters light having a wavelength of about 530 nm.

6. The component of claim 1, wherein the matrix comprises a hydrogel.

7. The component of claim 1, wherein the component is characterized as a contact lens.

8. The component of claim 1, wherein the component is characterized as an eyeglass lens.

9. The component of claim 1, wherein the MXene material comprises at least one of Mo₂TiC₂Tₓ, Ti₃C₂Tₓ, and Ti₂CTₓ.

10. The component of claim 1, wherein the MXene material is present at up to about 1 wt % of the weight of the matrix material, or up to about 0.5 wt % of the weight of the matrix material, or up to about 0.2 wt % of the weight of the matrix material.

11. A method, comprising fabricating a component according to claim 1.

12. A method, comprising the use of a component according to claim 1 by an individual experiencing color vision deficiency.

13. A method, comprising the use of a component according to claim 1 by an individual in need of protection from ultraviolet radiation.

14. A method, comprising contacting a MXene and a matrix material to form a color-active composition and forming the color-active composition into an ophthalmic component.

15. The method of claim 14, wherein the matrix material is porous.

16. The method of claim 14, wherein the matrix material comprises a hydrogel.

17. The method of claim 14, wherein the MXene is present at up to about 2 wt % of the matrix material in the composition.

18. The method of claim 17, wherein the MXene is present at up to about 1 wt % of the matrix material in the composition.

19. The method of claim 17, wherein the ophthalmic component is a contact lens.

20. The method of claim 17, wherein the ophthalmic component is a glasses lens, a visor, or a faceshield.
